SEQUENCE LISTING (4 sequences)

SEQ ID NO: 1 - protein, 666 aa, Artificial Sequence
  C5V
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 2 - protein, 485 aa, Artificial Sequence
  VC5
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSQVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSGGGGSGGGGSGGGGSDIQMTQSPSSLSASVGDRVTITCGASENIYGALNWYQQKPGKAPKLLIYGATNLADGVPSRFSGSGSGTDFTLTISSLQPEDFATYYCQNVLNTPLTFGQGTKVEIK

SEQ ID NO: 3 - protein, 15 aa, Artificial Sequence
  GS linker
GGGGSGGGGSGGGGS

SEQ ID NO: 4 - protein, 20 aa, Artificial Sequence
  signal peptide
METDTLLLWVLLLWVPGSTG

(19) United States
(12) Patent Application Publication
(10) Pub. No.: US 2020/0115443 A1
(43) Pub. Date: Apr. 16, 2020

(54) BI-FUNCTIONAL FUSION PROTEINS AND USES THEREOF

(71) Applicant: Trican Biotechnology Co., Ltd, New Taipei City (TW)

(72) Inventors: Huang-Tsu Chen, Cupertino, CA (US); Jiun-Shyang Leou, Hsinchu City (TW); Chung-Yuan Hsu, Kaohsiung City (TW); Cheng-Ke Li, Taoyuan City (TW); Yun-Ting Wang, New Taipei City (TW); Li-Tsen Lin, New Taipei City (TW)

(73) Assignee: Trican Biotechnology Co., Ltd, New Taipei City (TW)

(21) Appl. No.: 16/600,075

(22) Filed: Oct. 11, 2019

Related U.S. Application Data
(60) Provisional application No. 62/744,936, filed on Oct. 12, 2018.

(51) Int. Cl. C07K 16/22; C07K 16/28; C07K 14/71; A61P 27/02

(52) U.S. Cl. CPC C07K 16/22; C07K 16/28; C07K 14/71; A61P 27/02; C07K 2317/31; A61K 2039/505; C07K 2317/55; C07K 2317/622; C07K 2317/24; C07K 2319/70; C07K 2317/51

(57) ABSTRACT

The present invention provides a bi-functional fusion protein simultaneously targeting the complement and the vascular endothelial growth factor (VEGF). The bi-functional fusion proteins contain two or more domains of human proteins and are of all human sequences, and thus are expected to be non-immunogenic, and potentially can be used therapeutically in human targeting complement and VEGF related diseases.

### FIELD OF THE INVENTION

The present invention relates to bi-functional fusion proteins, in which the heavy chain of an anti-C5 antibody is fused with a VEGF trap, or the heavy chain of an anti-VEGF antibody Fab is fused with an anti-C5 antibody Scfv fragment.

### BACKGROUND OF THE INVENTION

Age-related macular degeneration (AMD) is the leading cause of blindness and visual impairment among the elderly (>50 years) in the United States and other developed countries (1). 85% of AMD are the dry (non-exudative) form in which cellular debris called drusen accumulates between the retina and the choroid. In the advanced dry AMD, central geographic atrophy occurs resulting loss of vision in the center of the eye. The wet (exudative or neovascular) form AMD is the more severe form in which abnormal blood vessels (choroidal neovascularization, CNV) grow up from the choroid through Bruch's membrane behind the macula, resulting in rapid vision loss. In recent years, increasing evidence has indicated that complement activation plays a major role in pathogenesis of AMD (2). High levels of complement proteins have been detected in drusen. Genetic studies have confirmed association of AMD risk and polymorphism in genes of complement proteins including Factor H (CFH), CFHR1, CFHR3, C2, C3, C5, Factor B, Factor I. In particular, CFH Y402H allele correlates highly with AMD risk. Increased levels of complement activation products have also been found in plasma of AMD patients. Consequently, several complement inhibitors are currently in clinical trials for treatment of AMD.

The complement system is functional effector of the innate immune system consisting of a number of plasma proteins and cell membrane proteins. Activation of the complement leads to a series of protease activation cascade triggering release of cytokines and amplification of the activation cascade. The end result of the complement activation is activation of the cell-killing membrane attack complex (MAC), inflammation caused by anaphylatoxins C3a and C5a, and opsonization of pathogens. The MAC, initiated through C5 cleavage, is essential for eliminating invading pathogens and damaged, necrotic, and apoptotic cells.

Delicate balance between defense against pathogen and avoidance of excess inflammation has to be achieved for the complement system (3). Many inflammatory, autoimmune, neurodegenerative and infectious diseases have been shown to be associated with excessive complement activities. Pathogenesis of Ischemia/reperfusion injury has indicated that the complement activation leads to inflammation-induced damage in a number of diseases, including Acute Myocardial Infarction, Stroke, Hemorrhagic and Septic Shock, and complication of coronary artery bypass graft surgery (4). Complement pathway seems to be a major contributor to a number of autoimmune diseases, including Systemic Lupus Erythematosus (5), Rheumatoid Arthritis, Psoriasis, and Asthma (6). Complement activation has also been correlated with the pathology of Alzheimer's disease (7) and other neurodegenerative diseases such as Huntington's disease, Parkinson's disease, and AMD (8).

The complement system can be activated through three different pathways: the classical pathway, the alternative pathway, and the lectin pathway (9). All three pathways go through critical protease complexes of C3-convertase and C5-convertase that cleave complement components C3 and C5, respectively. The classical pathway is initiated by binding of Clq to antibodies IgM or IgG leading to activation of the C1 complex that cleaves complement components C2 and C4, producing C2a, C2b, C4a, and C4b. C4b and C2b then forms the classical pathway C3-convertase, which promotes cleavage of C3 into C3a and C3b. C3b then forms the C5-convertase by binding to C4bC2b (the C3-convertase). The lectin pathway is identical to the classical pathway downstream of the C3-convertase and is activated by binding of mannose-binding lectin (MBL) to mannose residues on the pathogen surface. The MBL-associated serine proteases MASP-1 and MASP-2 can then cleave C4 and C2 to form the same C3-convertase as in the classical pathway. Unlike the classical and the lectin pathways that are specific immune responses requiring antigens, the alternative pathway is a non-specific immune response that is continuously active at a low level. Spontaneously hydrolysis of C3 leads to C3a and C3b. C3b can bind Factor B and then cleave Factor B to Ba and Bb with facilitation of factor D. The C3bBb complex which can be stabilized by binding of Factor P (Properdin) is the C3-convertase of the alternative pathway that cleaves C3 to C3a and C3b. C3b can join the C3bBb complex to form C3bBbC3b complex that is the C5-convertase of the alternative pathway. The C5-convertases from all three pathways can cleave C5 to C5a and C5b. The C5b then recruits and assembles C6, C7, C7, C8 and multiple C9 molecules to assemble the MAC. This creates a hole or pore in the membrane that can kill or damage the pathogen or cell.

Several monoclonal antibodies against complement proteins have been used as therapeutic agents (10). Eculizumab, a humanized antibody against C5 protein, has been approved to treat paroxysmal nocturnal hemoglobinuria (PNH) in 2007 (the patents will expire in the US on 16 Mar. 2021 and in Europe on 1 May 2020). Eculizumab was tested systemically to treat AMD in clinical. Though well tolerated in trials, Eculizumab did not decrease the growth rate of GA (an advanced form of AMD) significantly, in clinical. Possible explanations might be due to low Eculizumab dosage used, or direct intravitreal injection is needed for Eculizumab to achieve adequate level to function. Several anti-C5 antibodies, such as Pexelizumab and Tesidolumab, currently are tested in trials to treat Geographic Atrophy, Non-infectious Panuveitis, Exudative Macular Degeneration, Non-infectious Posterior Uveitis, and/or Age-related Macular Degeneration. Antibodies against C5a (TNX-558), Factor D (TNX-234), Factor P, and C3b have been developed and evaluated in various disease models. Additionally, an aptamer inhibitor of human C5 (ARC1905) and a 13-amino acid cyclic peptide (Compastatin) against C3 are been evaluated in clinical trials to treat AMD disease.

Vascular endothelial growth factor (VEGF) is one of the most important proteins that promote angiogenesis, which is a tightly regulated process of developing new blood vessels from a pre-existing vascular network (11). The human VEGF gene family contains 5 members: VEGF-AVEGF-B, VEGF-C, VEGF-D and placental growth factor (PIGF). In addition, multiple isoforms of VEGF-A, VEGF-B and PIGF are generated through alternative RNA splicing (12). VEGF-A is the prototypic member of the family and also the most studied member. VEGF-A has been shown to stimulate endothelial cell mitogenesis, promote cell survival and proliferation, induce cell migration, and increase microvascular permeability. All members of the VEGF family stimulate cellular responses by binding to cell surface VEGF receptors (VEGFRs). The VEGFR receptors are tyrosine kinase receptors that have extracellular regions consisting of 7 immunoglobulin (IG)—like domains. VEGFR-1 (Flt-1) binds VEGF-A, -B, and PIGF, and can function as a decoy receptor for VEGFs or a regulator of VEGFR-2. VEGFR-2 (KDR/Flk-1) binds all VEGF isoforms and is the predominant mediator of VEGF-induced angiogenesis signaling. VEGFR-3 (Flt-4) binds VEGF-C and VEGF-D, but not VEGF-A, and functions as a primary mediator of lymphangiogenesis.

Angiogenesis is required during development and normal physiological processes such as wound healing and the menstrual cycle and been proven to be involved in a number of disease pathogenesis, including AMD, RA, Diabetic Retinopathy, tumor growth and metastasis. Inhibition of angiogenesis has been shown to be effective in therapeutic applications. Several inhibitors against VEGF-A have been approved by FDA. For example, a humanized antibody against VEGF-A (Avastin), an antibody Fab fragment against VEGF-A (Lucentis), and a VEGF trap (Eylea). Avastin is approved to treat Metastatic Colorectal Cancer (mCRC), Non-Small Cell Lung Cancer (NSCLC), Glioblastoma (GBM), and Metastatic Kidney Cancer (mRCC). Lucentis and Eylea are approved to treat wet AMD. A number of other anti-VEGF molecules, such as Brolucizumab, Varisacumab and Conbercept are currently in clinical development.

### BRIEF SUMMARY OF THE INVENTION

The object of the present invention is to develop a therapeutic agent capable of treating various complement and VEGF-related diseases such as Age-related Macular Degeneration (AMD), and the like, by more effectively and simultaneously inhibiting complement and VEGF pathways to solve above-described problems, and as a result, find that a bi-functional fusion protein simultaneously targeting complement and VEGF effectively exhibits anti-complement and anti-VEGF efficacy.

The present invention provides a fusion protein that inhibits a complement signaling pathway and a VEGF signaling pathway, wherein the fusion protein contains a complement binding domain and a VEGF binding domain.

In one aspect, the invention provides a bi-functional fusion protein comprising one or more C5 binding motif containing fragments and one or more VEGF binding motif containing fragments, which are fused with a short flexible linker, thereby providing a significantly improved efficacy in inhibition of complement and angiogenesis simultaneously.

In one embodiment, the present invention provides a bi-functional fusion protein, C5V, simultaneously targeting the complement and the VEGF and providing a complement C5 cleavage blocking activity and an anti-angiogenesis efficacy concurrently, wherein C5 is a complement C5 binding motif, such as the heavy chain of Eculizumab; V is a VEGF binding motif, such as VEGFR1 ECD D2 and VEGFR2 ECD D3, or its chimeric domains; and a short flexible GS linker is inserted in between to ensure correct folding of each domain and minimal steric hindrance.

In another embodiment, the present invention provides a bi-functional fusion protein, VC5, simultaneously targeting the complement and the VEGF and providing a complement C5 cleavage blocking activity and an anti-angiogenesis efficacy concurrently, wherein V is a VEGF binding motif, such as the heavy chain of Ranibizumab Fab; C5 is a complement C5 binding motif, such as the Scfv of Eculizumab; and a short flexible GS linker is placed between the heavy chain and Scfv.

In yet other embodiment, the present invention provides a bi-functional fusion protein that is useful for treatment of complement and VEGF-related diseases.

Accordingly, the present invention also provides a pharmaceutical composition comprising the bi-functional fusion protein of the disclosure and a pharmaceutically acceptable carrier.

In one embodiment, the pharmaceutical composition is useful for treatment of complement and VEGF-related diseases

Further, the present invention provides a method for treating a complement and VEGF related disease in a subject in need thereof, comprising administering to said subject a therapeutically effective amount of the bi-functional fusion protein disclosed herein.

In one embodiment, the complement and VEGF related disease disclosed herein is selected from the group consisting of atherosclerosis, age-related macular degeneration, acute myocardial infarction (AMI), glomemephritis, asthma, thrombosis, deep vein thrombosis, multiple sclerosis, Alzheimer's disease, autoimmune uveitis, systemic lupus erythematosus (SLE), lupus nephritis, ulcerative colitis, inflammatory bowel disease, Crohn's disease, adult respiratory distress syndrome (ARDS), multiple sclerosis, diabetes mellitus, Huntington's disease, Parkinson's disease, rheumatoid arthritis, juvenile rheumatoid arthritis, osteoarthritis, psoriatic arthritis, CNS inflammatory disorders, myasthenia gravis, glomerulonephritis, and autoimmune thrombocytopenia, aneurysm, atypical hemolytic uremic syndrome, spontaneous fetal loss, recurrent fetal loss, traumatic brain injury, psoriasis, autoimmune hemolytic anemia, hereditary angioedema, stroke, hemorrhagic shock, septic shock, complication from surgery such as coronary artery bypass graft (CABG) surgery, pulmonary complications such as chronic obstructive pulmonary disease (COPD), ischemia-reperfusion injury, organ transplant rejection, multiple organ failure and cancer. Preferentially, the complement and VEGF related disease is age-related macular degeneration and cancer. More preferentially, the complement and VEGF related disease is age-related macular degeneration.

The details of one or more embodiments of the invention are set forth in the description below. Other features or advantages of the present invention will be apparent form the following drawings and detailed description of several embodiments, and also from the appended claims.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1 is schematic drawings of the bi-functional fusion proteins with complement C5 cleavage blocking activity and VEGF inhibiting activity, concurrently. The bi-functional fusion protein C5V was generated through fusing the heavy chain of Eculizumab with a VEGF inhibiting motif at its C-terminal. The VEGF binding motif used in this construct comprises the VEGFR1 D2 and VEGFR2 D3 chimeric domains (patents will expire in the US in 2020, and 2021 in European). The bi-functional fusion proteins VC5 was created by fusing the heavy chain Fd chain of Fab derived from Lucentis (the patents on Lucentis will expire in the US in June 2020 and in Europe in 2022 [1]) with Eculizumab (Scfv) at its C-terminal. Both fusion proteins contain a short GS linker between the functional entities to ensure flexibility and folding.

FIGS. 2A and 2B are SDS-PAGE gel analyses of the purified bi-functional fusion proteins C5V and VC5, respectively. 2 μg of protein was loaded in each lane. Lane 1 is the non-reducing condition; lane 2 is the reducing condition.

FIG. 3 is the direct in vitro binding of complement C5 using the purified bi-functional fusion proteins. The bound proteins, after washing, were detected with HRP-conjugated goat anti-human IgG Fc specific antibody for C5V, or HRP-conjugated goat anti-human Fab specific antibody for VC5.

FIG. 4 shows the direct in vitro binding of VEGF with the purified bi-functional fusion proteins. The bound proteins after washing were detected with HRP-conjugated goat anti-human IgG Fc specific antibody for C5V, and HRP-conjugated goat anti-human Fab specific antibody for VC5.

FIG. 5 is the affinity assessment of the bi-functional fusion proteins to VEGF-A in solution. After overnight incubation of bi-functional fusion proteins and VEGF in solution, the free VEGF concentration was determined by a sandwich ELISA assay.

FIG. 6 is inhibition of the alternative complement pathway by the purified bi-functional fusion proteins. Normal human serum was first incubated with various concentrations of the bi-functional fusion proteins and was then used to lyse rabbit erythrocytes in the presence of 5 mM of Mg²⁺ and 5 mM of EGTA. Hemolysis was detected by absorption at OD 412 nm.

FIG. 7 is the result of HUVEC cell growth inhibition assay. HUVECs were maintained in the Endothelial Cell Growth Medium (Lonza, Inc.) with 2% FBS. A 96-well flat bottom microtiter plate was coated with collagen, and then incubated with 50 μl of 1 nM of VEGF-A (R&D systems, USA) with various concentrations of fusion proteins. After incubation for 72 hours at 37C with 5% CO₂, cell proliferation was assayed by adding 10 μl of MTS detection reagent (Promega, USA) to each well and then measuring OD absorption at 450/650 nm.

FIG. 8 shows an inhibitory effect of the bi-functional fusion proteins C5V on VEGF-induced HUVEC cells tube formation. Quantification of endothelial network formation was performed by Image J angiogenesis system (5 images/sample) and is represented as fold change compared to VEGF treatment.

FIG. 9 shows an inhibitory effect of the bi-functional fusion protein C5V on VEGF-induced endothelial cell invasion.

FIG. 10A and 10B show the inhibition of laser-induced choroidal neovascularization (CNV) in mice by the bi-functional fusion protein C5V. FIG. 10A represents the vascular leakage in laser-induced CNV model. FIG. 10B represents the quantification of laser-induced CNV lesions.

### DETAILED DESCRIPTION OF THE INVENTION

It is to be understood that this disclosure is not limited to particular methods and experimental conditions described, as such methods and conditions may vary.

It is also to be understood that the terminology used herein is for the purpose of describing particular embodiments only, and is not intended to be limiting, since the scope of the present disclosure will be limited only by the appended claims.

Unless otherwise defined herein, scientific and technical terms used herein have the meanings that are commonly understood by those of ordinary skill in the art.

As used herein, the indefinite articles “a” and “an” and the definite article “the” are intended to include both the singular and the plural, unless the context in which they are used clearly indicates otherwise.

In certain aspects, the present invention relates to a bi-functional fusion protein that simultaneously targets the complement C5 and the VEGF pathway. Since the complement and VEGF pathways are implicated in a number of diseases including Age-related Macular Degeneration (AMD), a protein with bispecific inhibitory activities to complement and VEGF might provide significantly better therapeutic efficacies than proteins that inhibit either complement or VEGF, individually. In the invention, the said bi-functional fusion protein may be a bi-functional fusion protein C5V that is created through fusing the heavy chain of an anti-C5 antibody at its C-terminal with a VEGF inhibiting motif containing VEGFR1 extracellular domain 2 and VEGFR2 extracellular domain 3. On the other hand, the said bi-functional fusion protein may be a bi-functional fusion protein VC5 that is generated through fusing the Fd chain of an anti-VEGF antibody Fab with an anti-05 antibody Scfv fragment at its C-terminal. The bi-functional fusion proteins C5V and VC5 are shown to be able to bind the complement C5 protein and the VEGF with high affinities, and also be able to inhibit the functions of the complement and VEGF pathways in cell-based assays, respectively. The bi-functional fusion proteins contain the domains of human proteins and are of all human origins, and are expected to be non-immunogenic, and thus, potentially can be further developed as therapeutics for treating complement and angiogenesis involved diseases.

As used herein, the term “fusion protein” refers to a protein created through the connection of two or more binding proteins, or motifs, or peptides/amino acid fragments coding for different genes, the translation of which genes results in a single or multiple polypeptides with multi-functional properties derived from each of the original proteins. A fusion protein can include a protein conjugated to an antibody, an antibody conjugated to a different antibody, or an antibody conjugated to a Fab fragment.

As used herein, the term “complement” refers to any of the small proteins of the complement cascade, sometimes referred to in the literature as the complement system or complement cascade. Activation of the complement leads to a series of protease activation cascade triggering release of cytokines and amplification of the activation cascade, leading to the activation of the cell-killing membrane attack complex (MAC), inflammation caused by anaphylatoxins C3a and C5a, and opsonization of pathogens. The MAC, initiated through C5 cleavage, is essential for eliminating invading pathogens and damaged, necrotic, and apoptotic cells.

As used herein, the term “Fab” refers to a region on an antibody that binds to antigens. It is composed of a variable and constant domain of the light chain and a variable domain and the first constant domain of the heavy chain antibody.

As used herein, the term “linker” refers to an amino acid residue or fragment, or a polypeptide comprising two or more amino acid residues joined by peptide bonds that are used to link two peptides, polypeptides or proteins. The linker may be a Fc fragment, which is an immunoglobulin Fc region of a wild-type or a variant of any human immunoglobulin isotypes, subclasses, or allotypes thereof.

As used herein, the term “Scfv” refers to the single chain fragment variable consisting of variable regions of heavy (V_{II}) and light (V_{L}) chains, which are joined together by a flexible peptide linker that can be easily expressed in functional form in E. coli, allowing protein engineering to improve the properties of Scfv such as increase of affinity and alteration of specificity.

In the invention, any motif, peptide, protein, or fragment having blocking VEGF activities may be used, for example anti-VEGF antibodies, VEGF traps, or VEGF receptor (VEGFR) extracellular Ig domains such as D1-D7, in particular, VEGFR1 extracellular Ig domain 2 (ECD, D2), VEGFR2 extracellular Ig domain 3 (ECD, D3).

In the invention, any motif, peptide, protein, or fragment binding to complement C5 may be used, for example, the heavy chain or Scfv of Eculizumab.

Since complement and VEGF are implicated in several diseases, such as AMD, to block the cleavage of complement C5 and the VEGF activity simultaneously, a bi-functional fusion protein against complement C5 and VEGF is created in this invention for treatment thereof.

In one embodiment of the invention, a fragment having the heavy chain of Eculizumab is used to generate a bi-functional fusion protein C5V (SEQ ID NO: 1) with a VEGF trap at C-terminal, wherein a short flexible GS linker (SEQ ID NO: 3) is inserted in between to ensure correct folding of each domain and minimal steric hindrance. In related embodiments, the C5V fusion protein includes an amino acid sequence at least about 45%, about 50%, about 55%, about 60%, about 65%, about 70%, about 75%, about 80%, about 85%, about 90%, about 95%, or about 99% identical to SEQ ID NO: 1.

Preferably, the said VEGF trap of the bi-functional fusion proteins C5V contains a VEGFR1 ECD D2 and VEGFR2 ECD D3.

In another embodiment of the invention, a fragment containing a heavy chain of Ranibizumab Fab is fused at the C-terminal of a complement C5 binding motif to construct a bi-functional fusion protein VC5 (SEQ: ID NO 2) with a short flexible GS linker (SEQ ID NO: 3) between them to ensure that the folding is correct and steric hindrance is minimized. In related embodiments, the bi-functional fusion protein VC5 includes an amino acid sequence at least about 45%, about 50%, about 55%, about 60%, about 65%, about 70%, about 75%, about 80%, about 85%, about 90%, about 95%, or about 99% identical to SEQ ID NO: 2.

Particularly, the said complement C5 binding motif of the bi-functional fusion protein VC5 contains the Scfv fragment of Eculizumab.

In further embodiment, all fusion proteins described in this invention are leaded by a signal peptide (SEQ ID NO: 4) for the extracellular secretion of expressed proteins.

The resulting sequences of the bi-functional fusion proteins C5V and VC5 are shown in SEQ ID NO: 1 and SEQ ID NO: 2, respectively.

In the present invention, the above-mentioned bi-functional fusion proteins C5V and/or VC5 are transiently expressed by HEK293 cells and purified from the transfected cell culture supernatant via Protein G chromatography. It is found that the products having a purity greater than 90% are obtained in a single step purification process and all fusion proteins are properly formed and expressed.

In one embodiment of the present invention, the binding abilities of the bi-functional fusion proteins C5V or VC5 to the complement C5 are verified by using an ELISA binding assay. In certain embodiments, the bi-functional fusion proteins C5V or VC5 exhibit strong binding to the complement C5 protein with EC₅₀ 3.57 nM and 2.77 nM, respectively.

In another embodiment of the present invention, the binding abilities of the bi-functional fusion proteins C5V or VC5 to VEGF are verified by using an ELISA binding assay. In certain embodiments, the bi-functional fusion proteins C5V or VC5 exhibit strong binding to the VEGF-A protein with EC₅₀ 0.288 nM and 1.675 nM, respectively.

In yet other embodiment of the present invention, the binding affinities of the bi-functional fusion proteins C5V to VEGF in solution are determined by a competition binding assay. In the present invention, the C5V fusion protein binds to the VEGF-A protein with high affinity, and C5V has a higher binding affinity against VEGF-A protein than Eylea.

Assays known in the art and described herein (e.g., Examples 2-7) can be used for identifying and testing biological activities of the bi-functional fusion proteins C5V or VC5 of the present disclosure. In some embodiments, assays for testing the abilities of the bi-functional fusion proteins C5V or VC5 for inhibiting the complement pathway and VEGF-dependent HUVEC proliferation are provided.

Certain aspects of the present disclosure relate to the inhibitory activities of the bi-functional fusion proteins C5V or VC5 to the alternative complement pathway. Specifically, the bi-functional fusion proteins C5V or VC5 are incubated with normal human serum to inhibit the lysis of the rabbit erythrocytes in the presence of Mg²⁺ and EGTA. In certain embodiments of the present invention, the IC₅₀ of the erythrocyte hemolysis by C5V and VC5 are 25.31 nM and 36.65 nM, respectively.

In addition, the VEGF activities may be characterized by measuring the VEGF-dependent HUVEC cell growth. According to certain embodiments, the bi-functional fusion proteins C5V or VC5 and VEGFA are loaded into collagen pre-coated wells, and then HUVEC cells are allowed to be cultured herein. After incubation, the cell growth is analyzed by MTS assay, and the IC₅₀ of C5V and VC5 are 0.195 nM and 0.313 nM, respectively.

Accordingly, in one aspect, the present invention provides a pharmaceutical composition comprising the bi-functional fusion protein of the disclosure and a pharmaceutically acceptable carrier.

In some embodiments, the present invention provides a pharmaceutical composition for use in inhibiting the complement pathway. In some embodiments, the present invention provides a pharmaceutical composition for use in inhibiting VEGF signaling pathway. In some embodiments, the present invention provides a pharmaceutical composition for use in inhibiting complement activation and VEGF signaling pathway in a subject comprising administering to the subject an effective amount of the fusion protein to inhibit complement activation and VEGF signaling pathway.

In some embodiments, the pharmaceutical composition can be used for treatment of an complement and/or VEGF-related disease including, but not limited to, atherosclerosis, macular degeneration (e.g., age-related macular degeneration), acute myocardial infarction (AMI), glomernephritis, asthma, thrombosis, deep vein thrombosis, multiple sclerosis, Alzheimer's disease, autoimmune uveitis, systemic lupus erythematosus (SLE), lupus nephritis, ulcerative colitis, inflammatory bowel disease, Crohn's disease, adult respiratory distress syndrome (ARDS), multiple sclerosis, diabetes mellitus, Huntington's disease, Parkinson's disease, rheumatoid arthritis, juvenile rheumatoid arthritis, osteoarthritis, psoriatic arthritis, CNS inflammatory disorders, myasthenia gravis, glomerulonephritis, and autoimmune thrombocytopenia, aneurysm, atypical hemolytic uremic syndrome, spontaneous fetal loss, recurrent fetal loss, traumatic brain injury, psoriasis, autoimmune hemolytic anemia, hereditary angioedema, stroke, hemorrhagic shock, septic shock, complication from surgery such as coronary artery bypass graft (CABG) surgery, pulmonary complications such as chronic obstructive pulmonary disease (COPD), ischemia-reperfusion injury, organ transplant rejection, multiple organ failure and cancer. In some embodiments, the cancer that can be treated or prevented by the fusion proteins described herein includes colorectal cancer, metastatic colorectal cancer, non-small cell lung cancer, lymphoma, leukemia, adenocarcinoma, glioblastoma, kidney cancer, metastatic kidney cancer, gastric cancer, prostate cancer, retinoblastoma, ovarian cancer, endometrial cancer, and breast cancer.

In some embodiments, the pharmaceutical composition can be used for treatment of an ocular disease including, but not limited to, wet age-related macular degeneration, dry age-related macular degeneration, diabetic retinopathy, diabetic retinal edema, diabetic macular edema, retrolental fibroplasias, retinal central occlusion, retinal vein occlusion, ischemic retinopathy, hypertensive retinopathy, uveitis (e.g., anterior, intermediate, posterior, or panuveitis), Behcet's disease, Biett's crystalline dystrophy, blepharitis, glaucoma (e.g., open-angle glaucoma), neovascular glaucoma, neovascularization of the cornea, choroidal neovascularization (CNV), subretinal neovascularization, corneal inflammation, and complications from corneal transplantation.

In various embodiments, complement and angiogenesis involved diseases may be AMD.

As used herein, the term “Age-related Macular Degeneration (AMD)” is a serious eye condition that blurs the sharp, central vision as needed for “straight-ahead” activities such as reading, sewing and driving. Normally, AMD affects the macula, the part of an eye. Most AMD are the dry form with cellular debris accumulating between the retina and the choroid. In the advanced dry AMD, central geographic atrophy occurs resulting loss of vision in the center of the eye. The wet form AMD is the more severe form in which abnormal blood vessels (choroidal neovascularization, CNV) grow up from the choroid through Bruch's membrane behind the macula. These blood vessels leak blood and fluid into the retina, causing distortion of vision that makes straight lines look wavy, as well as blind spots and loss of central vision. These abnormal blood vessels eventually scar, leading to permanent loss of central vision.

The pharmaceutical composition according to present invention can be formulated into a suitable form containing the bi-functional fusion protein alone or together with a pharmaceutically acceptable carrier, and may further contain an excipient or a diluent. The carrier can be solvents, dispersion media, isotonic agents and the like. The carrier can be liquid, semi-solid or solid carriers. In some embodiments, carriers may be water, saline solutions or other buffers (such as serum albumin and gelatin), carbohydrates (such as monosaccharides, disaccharides, and other carbohydrates including glucose, sucrose, trehalose, mannose, mannitol, sorbitol, or dextrins), gel, lipids, liposomes, resins, porous matrices, binders, fillers, coatings, stabilizers, preservatives, antioxidants (including ascorbic acid and methionine), chelating agents (such as EDTA), salt forming counter-ions (such as sodium), non-ionic surfactants [such as TWEEN™, PLURONICS™ or polyethylene glycol (PEG)], or combinations thereof.

The pharmaceutical composition of the present invention may be administrated to mammals including humans by any method. For example, the composition of the present invention may be administrated orally or parietally. The parietal administration may be, but is not limited to, intravenous, intra-muscular, intra-arterial, intramedullary, intradural, intracardiac, transdermal, subcutaneous, intraperitoneal, intranasal, intestinal, topical, sublingual, or rectal administration.

The pharmaceutical composition can contain more than one additional beneficial compound for preventing or treating complement and/or VEGF-associated diseases.

In some embodiments, the pharmaceutical composition can comprise more than one additional therapeutic agent for treating said disease or disorder to be treated. For example, the additional agent may be an anti-dyslipidemic agent, a PPAR-α agonist, a PPAR-β agonist, a PPAR-γ agonist, an anti-amyloid agent, an inhibitor of lipofuscin, a visual-light cycle modulator, an antioxidant, a neuroprotector, an apoptosis inhibitor, a necrosis inhibitor, a C-reactive protein inhibitor, an inhibitor of inflammasomes, an anti-inflammatory agent, an immunosuppressant, a modulator of matrix metalloproteinase, an inhibitor of the complement system or components, and an anti-angiogenic agent.

The invention is further illustrated by the following example, which should not be construed as further limiting.

### Example 1

### Expression and Purification of Bi-Functional Fusion Proteins that Inhibited Both Complement and VEGF Pathways

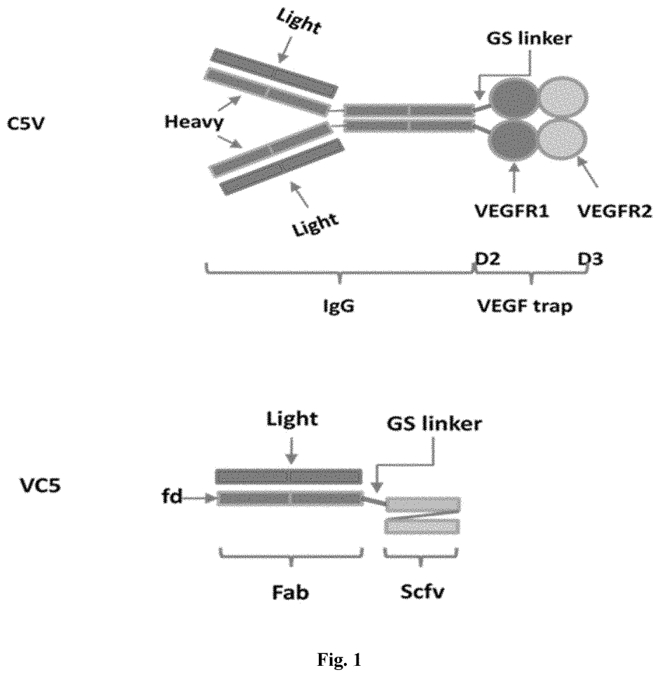

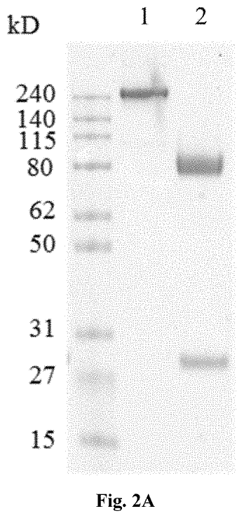

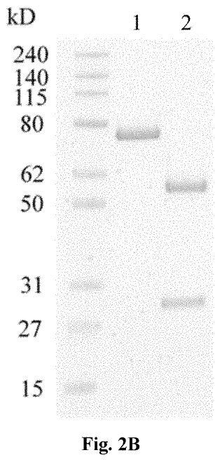

Antibody or antibody fragment with activity to bind and inhibit complement C5 cleavage can be used as complement activation blocker. Anti-VEGF antibody fragment or VEGF trap can be used as VEGF inhibiting motif. The cDNAs were synthesized and used to generate bi-functional expression vectors. The complement C5 cleavage blocker can be placed at either end, N-terminal or the C-terminal, of the VEGF inhibiting motif as shown in FIG. 1. Fusion proteins contained a GS linker between functional entities and were leaded with a signal peptide at the N-terminal for secretion out of the cells. Purified expression vectors were used to transfect HEK293 cells transiently, and cell culture media were harvested after 96 hours of incubation and purified via Protein G chromatography. 2 μg of purified bi-functional fusion proteins were PAGE-analyzed under reducing and non-reducing conditions (FIG. 2A and 2B). Purities of the 1-step purification is greater than 90% in both cases.

### Example 2

### In Vitro Binding of Bi-Functional Fusion Proteins to Complement C5 and VEGF

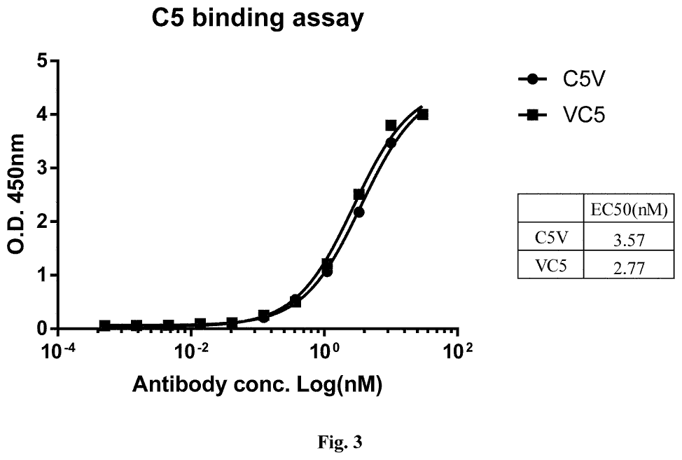

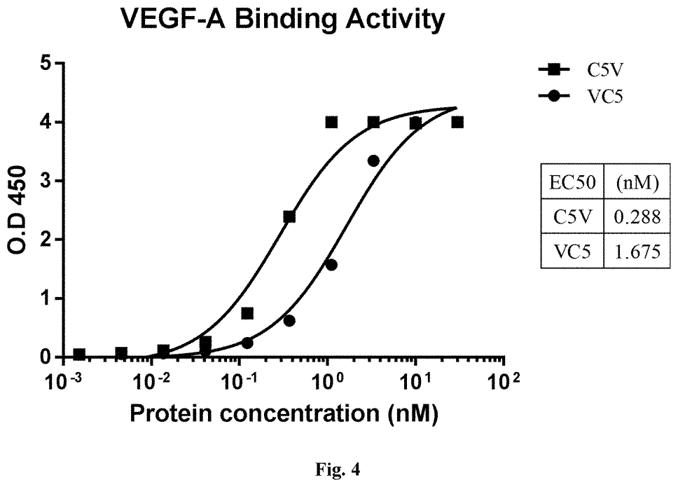

To test direct binding of purified fusion protein against complement C5 or VEGF in ELISA, C5 or VEGF-A pre-coated wells (100 ng/well) were incubated with 0-30 nM of purified proteins for 1 hour. After washing, 1:2500 dilution of HRP-conjugated anti-human Fc antibody (Jackson Immunochemicals, USA) was added to each well for another 1 hour of incubation. After final washing, TMB reagent (ThermoFisher, USA) was added and OD absorption at 450 nm was measured and data were analyzed by sigmoidal curve fitting using Prism 4. As shown in FIG. 3, the bi-functional fusion proteins C5V and VC5 exhibited strong binding to C5 with EC₅₀ 3.57 nM and 2.77 nM, respectively. The bi-functional fusion proteins C5V and VC5 also exhibited strong binding to VEGF-A with EC₅₀ of 0.288 nM and 1.675 nM, respectively (FIG. 4).

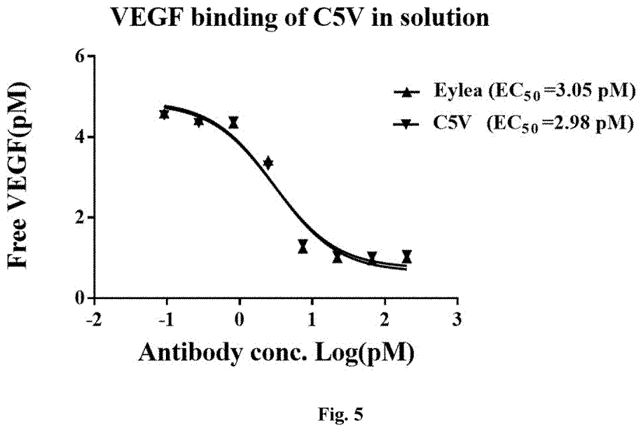

To better assess the binding affinity of fusion proteins to VEGF in solution, 5 pM of VEGF-A (R&D Systems, Inc.) was incubated with 0-100 pM of purified proteins for overnight. Next day, free VEGF concentrations were determined using DEV00 kit (R&D Systems, USA). The data analyzed by sigmoidal curve fitting, accordingly. As shown in FIG. 5, the bi-functional fusion proteins C5V binds to the VEGF-A protein with high affinity, and C5V has a higher binding affinity against VEGF-A protein than Eylea.

### Example 3

### Inhibition of the Alternative Complement Pathway by Bi-Functional Fusion Proteins C5V and VC5

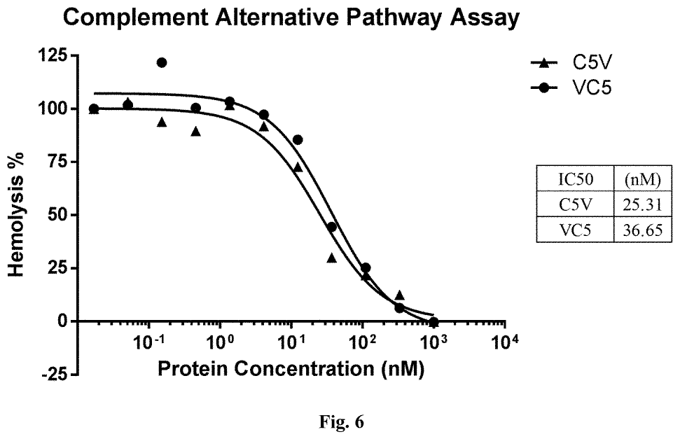

Activation of the alternative pathway of complement requires only Mg²⁺, whereas the classical and lectin complement pathways require both Ca²⁺ and Mg²⁺ ions. Thus, the alternative complement activity can be assayed in the presence of classical pathway proteins when 5 mM of Mg²⁺ and 5 mM of EGTA are included in the assays, in which EGTA chelates Ca²⁺ preferentially. The hemolysis assay can used to assess the inhibition of fusion proteins to the alternative complement activation. In the experiment, the dilution of normal human serum (CompTech, USA) that lysed 90% of 1.25×10⁷ rabbit erythrocytes/ml (Er, Complement Technology, Inc.) was first determined after 30 minutes incubation at 37° C. The assay was carried out in GVB⁰ buffer (0.1% gelatin, 5 mM Veronal, 145 mM NaCl, 0.025% NaN₃, pH 7.3) containing 5 mM of MgCl₂ and 5 mM of EGTA. Inhibition of the alternative complement pathway was initiated by mixing the dilution of normal human serum that can lyse 90% of Er with 0-500 nM of purified fusion proteins C5V and VC5 for 1 hour at 37° C. Hemolysis of Er was then assayed after 30 minutes incubation of the serum and Er. The data were analyzed using Prism 4. Results in FIG. 6 indicate that the bi-functional fusion proteins C5V and VC5 have IC₅₀ of 25.31 nM and 36.65 nM, respectively, for complement alternative pathway.

### Example 4

### Inhibition of VEGF-Dependent HUVEC Proliferation Assay by Angiogenesis Blockers

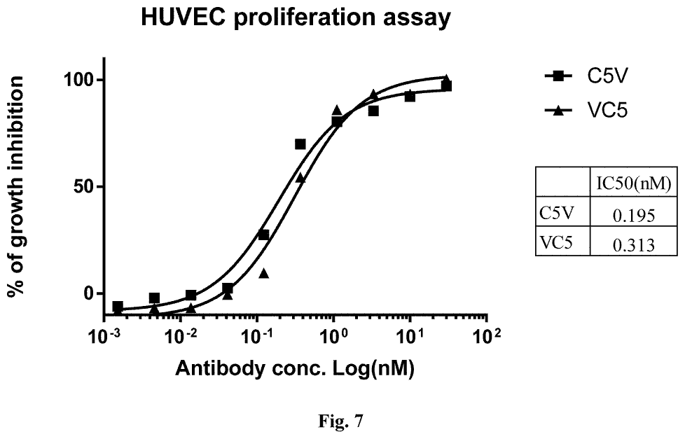

Purified bi-functional fusion proteins C5V and VC5 were used to inhibit VEGF activities in a cell base assay. Human Umbilical Vein Endothelial Cells (HUVEC cells, Lonza, USA) are commonly used to demonstrate VEGF-dependent cell proliferation which can be inhibited by VEGF blocker. In the experiment, HUVECs are maintained in the Endothelial Cell Growth Medium (Lonza, USA) with 2% FBS. Collagen pre-coated wells were loaded with 50 μl of 1 nM of VEGF-A (R&D systems, Inc.) and various concentrations of C5V or VC5 per well for 1 hour at 37° C. before adding 50 ul of HUVECs at 1×10⁵ cells/ml in Medium-199 (10% FBS, Hyclone, USA) to each well. After 72 hours incubation at 37° C. with 5% CO₂, cell growth was assayed by adding 10 μl MTS detection reagent (Promega, USA) to each well and then measuring OD absorption at 450/650 nm. As shown in FIG. 7, the abilities of the bi-functional fusion proteins C5V or VC5 exhibited good abilities for inhibiting VEGF-dependent HUVEC proliferation with IC₅₀ of 0.195 nM and 0.313 nM, respectively.

### Example 5

### Inhibition of VEGF-Induced Endothelial Cell Tube Formation by Bi-Functional Fusion Protein C5V

To examine the function of C5V in angiogenesis, an in vitro Matrigel tube formation assay was performed in human umbilical vein endothelial cells. HUVEC cells were incubated in basal media without serum for 2 hr, and then trypsinized by accutase. 4×10³ HUVEC cells were seeded onto Matrigel pre-coated wells containing VEGF (1 μg/ml) with Eylea (100 μg/ml) or C5V protein (100 μg/ml) at 37° C. for 4.5 hr. Quantification of endothelial network formation was performed by Image J angiogenesis system (5 images/sample).

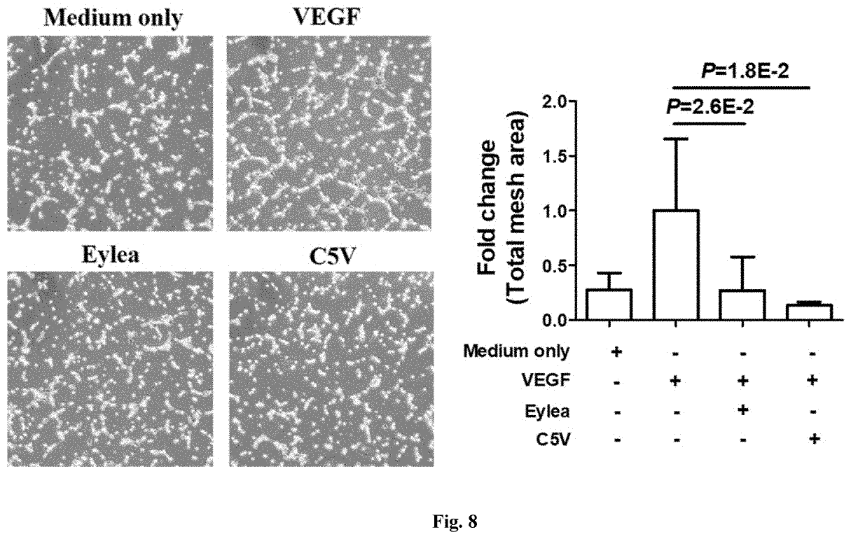

Under VEGF treatment, when cultured on Matrigel for 4.5 hr, HUVEC cells displayed a primary a vascular tubular network. However, the bi-functional fusion protein C5V disrupted the formation of tubular structures (FIG. 8A & 8B).

### Example 6

### Inhibition of VEGF-Induced Endothelial Cell Invasion by Bi-Functional Fusion Protein C5V

The invasive effect of bi-functional fusion proteins C5V on VEGF stimulation was examined by transwell analysis. The invasion assay was performed by precoating the transwell inserts with Matrigel Basement Membrane Matrix (BD Biosciences, San Diego, Calif., USA), according to the manufacturer's instructions. HUVEC cells (2×10⁵) with Medium-199 were placed in the upper well. The ligand human VEGF (0.2 μg/ml) were mixed with Eylea (2 μg/ml) or C5V (2 μg/ml) in the lower chamber, individually. The transwell plate were incubated for 24 h in a 5% incubator to allow cells from the upper well to transmigrate towards the bottom chamber. The membrane inserts were then fixed and stained with 1% crystal violet. Cells adhered to the lower surface of membrane inserts were visualized via microscopy and the average number of migrated cells was calculated using ImageJ software.

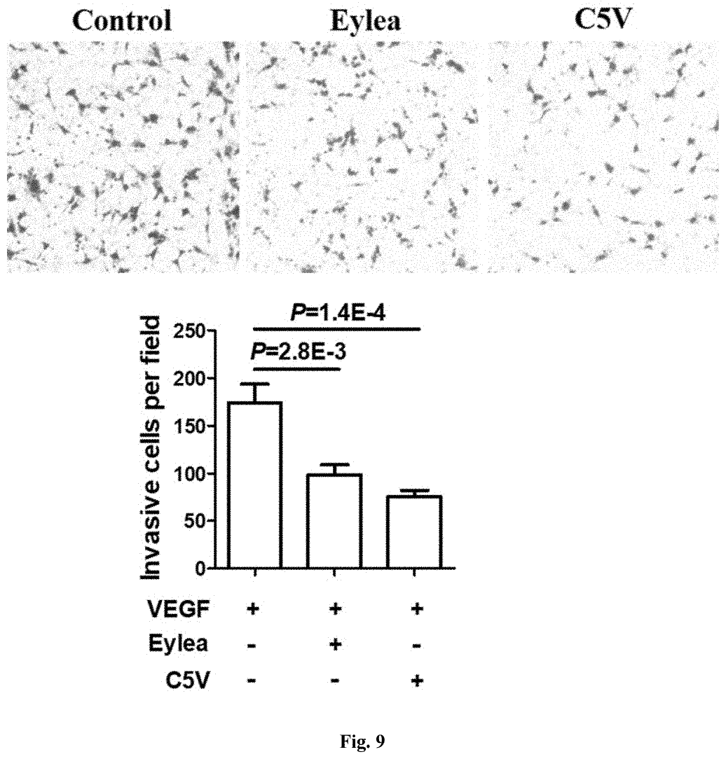

As shown in FIG. 9A and 9B, the bi-functional fusion protein C5V significantly inhibited the HUVEC cell invasion induced by VEGF.

### Example 7

### Bi-Specific Protein C5V Inhibits Neovascularization in Laser-Induced CNV Mouse Model

VEGF and C5 appear strongly linked to pathological neovascularization and vascular permeability, which are the hallmarks of ocular neovascular disease. Higher levels of C5 also correlate with disease severity in human wet Age-related Macular Degeneration (AMD) and inflammation. We generated an innovative design to co-targeting VEGF and C5. We tested the effect of the bi-specific protein C5V on angiogenesis by laser-induced choroidal neovascularization mouse model. In the laser-induced CNV model, male C57BL/6 mice were intravitreally injected with vehicle control, Eylea (40 μg) or C5V (40 μg) in the right eye on day 1 (n=5/group). The laser-induced damage to Bruch's membrane was identified of a bubble at the site of laser application. For fundus fluorescein angiography (FFA) analysis, anesthetized animals were intraperitoneally injected with 5% sodium fluorescein. Images were captured using the Micron III retinal imaging microscope (Phoenix Research Laboratories, San Ramon, Calif., USA). OCT images were acquired at 21 days poster-laser and neovascular lesion volume was measured as an ellipsoid. Ellipsoid volume was calculated by the formula V=4/3πabc where a (width), b (depth) and c (length) are the radii of the horizontal plane or vertical plane of the ellipsoid.

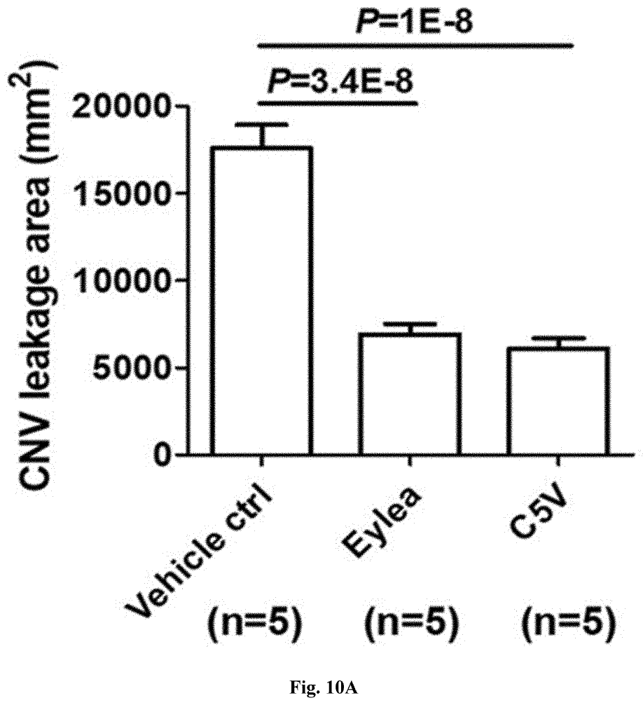

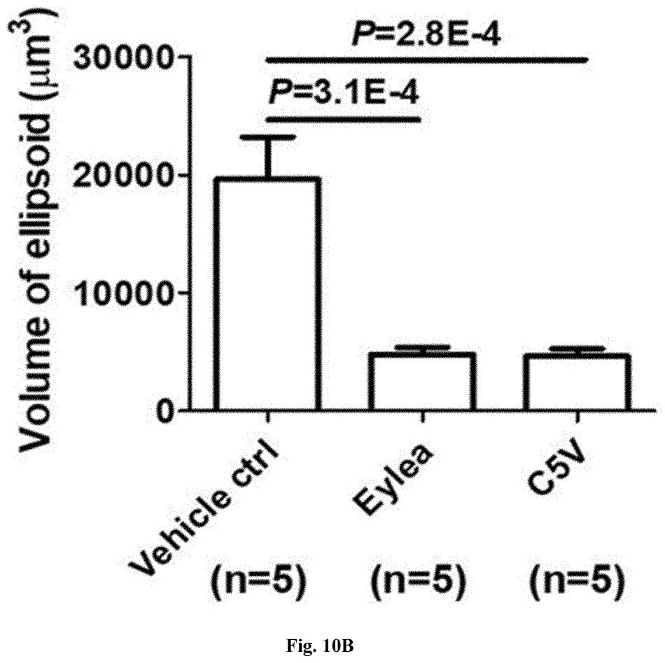

As shown in FIG. 10A, the bi-functional fusion protein C5V reduced vascular leakage in laser-induced CNV model (FIG. 10A). Quantification of laser-CNV lesions as ellipsoids by optical coherence tomography (OCT) showed a significant reduction in lesion volume after the bi-functional fusion protein C5V treatment (FIG. 10B).

While the present invention has been disclosed by way preferred embodiments, it is not intended to limit the present invention. Any person of ordinary skill in the art may, without departing from the spirit and scope of the present invention, shall be allowed to perform modification and embellishment. Therefore, the scope of protection of the present invention shall be governed by which defined by the claims attached subsequently.

### REFERENCES

1. Bird, A. C., “Therapeutic targets in age-related macular disease”, 2010, J. Clin. Invest., 120(9): 3033-3041
2. Issa, P. C., et al, “The significance of the complement system for the pathogenesis of age-related macular degeneration—current evidence and translation into clinical application”, 2011, Graefes. Arch. Clin. Exp. Ophthalmol., 249: 163-174
3. Ricklin, D., et al., “Complement-targeted therapeutics”, 2007, Nature Biotechnology, 25(11): 1265-1275
4. Markiewski, M. M., et al, “The role of complement in inflammatory diseases—from behind the scenes into the spotlight”, 2007, Am. J. Pathol. 171: 715-727
5. Manderson, A. P., et al, “The role of complement in the development of systemic lupus erythematosus”, 2004, Annul. Rev. Immunol. 22: 431-456
6. Guo, R. F., et al, “Role of C5a in inflammatory responses”, 2005, Annul. Rev. Immunol. 23: 821-852
7. Bonifati, D. M., et al, “Role of complement in neurodegeneration and neuroinflammation”, 2007, Mol. Immunol. 44: 999-1010
8. Gehrs, K. M., “Complement, Age-Related Macular Degeneration and a Vision of the Future”, 2010, Arch. Ophthalmol., 128 (3): 249-258
9. Wagner, E., et al., “Therapeutic potential of complement modulation”, 2010, Nat. Rev. Drug Discovery, 9(1): 43-56
10. Ehrnthaller, C., et al, “New Insights of an Old Defense System: Structure, Function, and Clinical Relevance of the Complement System”, 2011, Mol. Med., 17: 317-329
11. Ferrara, N., “Vascular Endothelial Growth Factor: Basic Science and Clinical Progress”, 2004, Endocrine Reviews, 25(4): 581-611
12. Sullivan L. A., et al, 2010, “The VEGF family in cancer and antibody-based strategies for their inhibition”, MAbs, 2(2): 165-175
13. Derbyshire M. Patent expiry dates for biologicals: 2017 update, 2018, Generics and Biosimilars Initiative Journal, 7(1):29-34.

1. A bi-functional fusion protein that inhibits a complement signaling pathway and a vascular endothelial growth factor (VEGF) signaling pathway, or both, wherein the fusion protein contains a complement C5 cleavage blocker, a VEGF inhibiting motif, and a GS linker at junction.

2. A bi-functional fusion protein comprising one or more complement C5 binding motif containing fragments and one or more VEGF binding motif containing fragments, which are fused with a linker, thereby providing a significantly improved efficacy in inhibition of complement and angiogenesis simultaneously.

3. The bi-functional fusion protein according to claim 2, wherein a complement C5 binding motif is used to generate the said bi-functional fusion protein with a VEGF trap at C-terminal, and a short linker is inserted in between.

4. The bi-functional fusion protein according to claim 3, wherein the complement C5 binding motif is the heavy chain of Eculizumab.

5. The bi-functional fusion protein according to claim 3, wherein the VEGF binding motif containing VEGFR1 ECD D2 and VEGFR2 ECD D3 chimeric domains.

6. The bi-functional fusion protein according to claim 3, wherein the short linker is a short flexible GS linker.

7. The bi-functional fusion protein according to claim 6, wherein the short flexible GS linker has the amino acid sequence set forth in SEQ ID NO: 3.

8. The bi-functional fusion protein according to claim 3, which has the amino acid set forth in SEQ ID NO: 1.

9. The bi-functional fusion protein according to claim 2, wherein a VEGF binding motif is fused at the C-terminal of a complement C5 binding motif to construct the said bi-functional fusion protein with a short linker between them.

10. The bi-functional fusion protein according to claim 9, wherein the VEGF binding motif is a fragment containing a heavy chain of Ranibizumab Fab.

11. The bi-functional fusion protein according to claim 9, wherein the complement C5 binding motif is the Scfv fragment of Eculizumab.

12. The bi-functional fusion protein according to claim 9, wherein the short linker is a short flexible GS linker.

13. The bi-functional fusion protein according to claim 12, wherein the short flexible GS linker has the amino acid set forth in SEQ ID NO: 3.

14. The bi-functional fusion protein according to claim 9, which has the amino acid set forth in SEQ ID NO: 2.

15. A pharmaceutical composition for the treatment of a complement and VEGF related disease, comprising a fusion protein or fragment of claim 1, and a pharmaceutically acceptable carrier.

16. The pharmaceutical composition according to claim 15, wherein the complement and VEGF related disease is selected from the group consisting of atherosclerosis, age-related macular degeneration, acute myocardial infarction (AMI), glomernephritis, asthma, thrombosis, deep vein thrombosis, multiple sclerosis, Alzheimer's disease, autoimmune uveitis, systemic lupus erythematosus (SLE), lupus nephritis, ulcerative colitis, inflammatory bowel disease, Crohn's disease, adult respiratory distress syndrome (ARDS), multiple sclerosis, diabetes mellitus, Huntington's disease, Parkinson's disease, rheumatoid arthritis, juvenile rheumatoid arthritis, osteoarthritis, psoriatic arthritis, CNS inflammatory disorders, myasthenia gravis, glomerulonephritis, and autoimmune thrombocytopenia, aneurysm, atypical hemolytic uremic syndrome, spontaneous fetal loss, recurrent fetal loss, traumatic brain injury, psoriasis, autoimmune hemolytic anemia, hereditary angioedema, stroke, hemorrhagic shock, septic shock, complication from surgery such as coronary artery bypass graft (CABG) surgery, pulmonary complications such as chronic obstructive pulmonary disease (COPD), ischemia-reperfusion injury, organ transplant rejection, multiple organ failure and cancer.

17. The pharmaceutical composition according to claim 16, wherein the complement and VEGF related disease is an age-related macular degeneration.

18. A method for treating a complement and VEGF related disease in a subject in need thereof, comprising administering to said subject a therapeutically effective amount of the bi-functional fusion protein of claim 1, wherein the complement and VEGF related disease is selected from the group consisting of atherosclerosis, age-related macular degeneration, acute myocardial infarction (AMI), glomernephritis, asthma, thrombosis, deep vein thrombosis, multiple sclerosis, Alzheimer's disease, autoimmune uveitis, systemic lupus erythematosus (SLE), lupus nephritis, ulcerative colitis, inflammatory bowel disease, Crohn's disease, adult respiratory distress syndrome (ARDS), multiple sclerosis, diabetes mellitus, Huntington's disease, Parkinson's disease, rheumatoid arthritis, juvenile rheumatoid arthritis, osteoarthritis, psoriatic arthritis, CNS inflammatory disorders, myasthenia gravis, glomerulonephritis, and autoimmune thrombocytopenia, aneurysm, atypical hemolytic uremic syndrome, spontaneous fetal loss, recurrent fetal loss, traumatic brain injury, psoriasis, autoimmune hemolytic anemia, hereditary angioedema, stroke, hemorrhagic shock, septic shock, complication from surgery such as coronary artery bypass graft (CABG) surgery, pulmonary complications such as chronic obstructive pulmonary disease (COPD), ischemia-reperfusion injury, organ transplant rejection, multiple organ failure and cancer.

19. The method of claim 18, wherein the complement and VEGF related disease is an age-related macular degeneration.

20. A pharmaceutical composition for the treatment of a complement and VEGF related disease, comprising a fusion protein or fragment of claim 2, and a pharmaceutically acceptable carrier.
